(19) United States
(12) Patent Application Publication
(10) Pub. No.: US 2007/0162089 A1
(43) Pub. Date: Jul. 12, 2007

(54) Cross-band communications in an implantable device

(75) Inventor: Oleg Mosesov, Maple Grove, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/328,758

(22) Filed: Jan. 9, 2006

(51) Int. Cl. A61N 1/00

(52) U.S. Cl. 607/60

(57) ABSTRACT

Embodiments of the invention provide cross-band communications with an implantable medical device. In one embodiment, an implantable medical device to be implanted into a body of a patient comprises one or more sensors configured to monitor physiological condition of the body of the patient; sensor electronics configured to process a signal received from the one or more sensors; a receiver configured to receive information from the external device in a first frequency band; a transmitter configured to sending information to an external device using a second frequency band which is different from the first frequency band; and a power supply. The receiver is powered on continuously when the implantable medical device is implanted into the body of the patient.

### CROSS-REFERENCES TO RELATED APPLICATIONS

Not Applicable

### BACKGROUND OF THE INVENTION

The present invention is directed to implantable medical devices and their use in monitoring physiological parameters within a patient's body. More particularly, the invention provides a system and a method for cross-band communications between an implantable medical device and an external device with increased power savings and improved response time.

The use of implantable medical devices has become increasingly commonplace as an effective method of monitoring the state and condition of a living body. An implantable medical device can be implanted within a human or an animal to monitor physiological parameters about the patient's well being. By being implanted directly within the body, implantable medical devices can provide continuous monitoring of the patient's condition without requiring continuous on site care by a caregiver or a physician. Implantable medical devices can also provide therapy within the body to change or improve the patient's physical state based on the physiological parameters received from sensors or the like. Implantable devices have been used to help treat a variety of physical disorders, such as heart disease, deafness, and diabetes with a large degree of success.

It is often desirable for such an implanted medical device to wirelessly communicate with a remote external device. For example, the implantable medical device may communicate the acquired physiological parameters to the external device for processing or display for other user output. The implantable medical device may also communicate to the remote device information about how the implantable medical device is configured, or the implantable medical device instructions for performing subsequent commands within the implantable medical device. Implantable medical devices typically use a predetermined frequency band to communicate information to and from the external device or programmer. One example of such a frequency band is the medical implant communication service (MICS) band, which operates between 402-405 MHz. The range of communication between the implantable medical device and the external device can be limited by a number of factors, including the limitations on the physical size of antennas that can be used within implantable device and signal loss due to transmission through the body of the patient. A typical range of communication is 2 meters or less.

The wireless communications to and from the implantable medical device are sent via the same frequency band, for example, the MICS frequency band. The MICS band can be split up into ten channels for transmission in the 402-405 MHz range. Regulations regarding the MICS band require the ten channels to be scanned through for the channel with the lowest ambient signal level to be transmitted on, or on the first available channel with an ambient signal below a given threshold. The scanning is typically performed by an external device and the selected channel is then communicated to the implantable medical device.

As wireless transmissions are sent between the implantable medical device and the external device, they can consume a significant amount of power during their operation. Implantable medical devices typically use an internal battery to power the device. The battery life or operational time that the implantable medical device can be used is an important factor in the design of the devices as a shortened battery life may require additional surgery to replace or recharge the device at an unwanted time for the patient. For this reason, it is desirable to reduce the power consumption within the implantable medical device to increase its time duration of operation.

Because of the power requirements needed to continuously sustain an implantable medical device, the implantable medical device may use a sleep state where the device is kept in a low-current usage state. The implantable medical device periodically looks or “sniffs” for a wake-up signal from an external device. Upon receiving the wake-up signal, the implantable medical device can be powered on to normal operation which utilizes significantly more current than during the sleep state. Alternatively, a duty cycle mode can be used by an implantable medical device to achieve lower power consumption, where the device is turned on during operation for a short time period and turned off following operation. Power savings can be achieved by duty cycling in that the implantable device is not continuously on. However, the latency of the implantable medical device is also increased in that the device cannot respond as quickly when powered off.

### BRIEF SUMMARY OF THE INVENTION

The present invention is directed to implantable medical devices and their use in monitoring physiological parameters within a patient's body. More particularly, the invention provides a system and a method for cross-band communications between an implantable medical device and an external device with increased power savings and an improved response time.

In accordance with an aspect of the present invention, an implantable medical device to be implanted into a body of a patient comprises one or more sensors configured to monitor physiological condition of the body of the patient; sensor electronics configured to process a signal received from the one or more sensors; a receiver configured to receive information from the external device in a first frequency band; a transmitter configured to sending information to an external device using a second frequency band which is different from the first frequency band; and a power supply. The receiver is powered on continuously when the implantable medical device is implanted into the body of the patient.

In some embodiments, the receiver is an ultra-low power receiver. The ultra-low power receiver uses a current of about 3 microamps or less. A receiving range of the receiver is at least about 2 meters. The one or more sensors comprise ECG sensors.

In specific embodiments, the second frequency band is higher than the first frequency band. The first frequency band may be an RFID frequency in the range of about 125 to about 134 kHz. The second frequency band may be a MICS band in the range of 402-405 MHz, or a band in the range of about 902 to about 928 MHz, or any other allowed frequency band The information received by the receiver from the external device comprises a selection of a frequency in a MICS band for the transmitter to send information to the external device. The receiver and transmitter are integrated as a transceiver.

Another aspect of the invention is directed to a method for communicating with an implantable medical device to be implanted into a body of a patient, which includes one or more sensors configured to monitor physiological condition of the body of the patient and sensor electronics configured to process a signal received from the one or more sensors. The method comprises receiving a first communication from an external device by a receiver in the implantable medical device within a first frequency band; sending a second communication from the implantable medical device to the external device within a second frequency band which is different from the first frequency band; and providing a power supply in the implantable medical device, wherein the receiver is powered on continuously when the implantable medical device is implanted into the body of the patient.

In some embodiments, the first communication is received by a receiver in the implantable medical device, the receiving being an ultra-low power receiver. The method may further comprise sensing ECG signals in the body of the patient. The implantable medical device may include a transceiver to receive the first communication and send the second communication.

In accordance with another aspect of the present invention, an implantable medical device to be implanted into a body of a patient comprises one or more sensors configured to monitor physiological condition of the body of the patient; sensor electronics configured to process a signal received from the one or more sensors; a receiver configured to receive information from the external device in a first frequency band; a transmitter configured to sending information to an external device using a second frequency band; and a power supply. The receiver is powered on continuously when the implantable medical device is implanted into the body of the patient. The first frequency band is lower than about 1 MHz.

In some embodiments, the first frequency band may be an RFID frequency in the range of about 125 and about 134 kHz. The second frequency band is higher than the first frequency band. The second frequency band may be in a range of 10 MHz and higher.

### BRIEF DESCRIPTION OF THE DRAWINGS

FIG. 1 is a simplified exemplary diagram showing wireless communications between an implantable medical device and an external device according to a specific embodiment of the invention.

FIG. 2 is a simplified exemplary flowchart of communications between an implantable medical device and an external device according to a specific embodiment of the present invention.

FIG. 3 is a simplified exemplary diagram of an implantable medical device according to a specific embodiment of the present invention.

FIG. 4 is a simplified exemplary diagram illustrating communications between an implantable medical device and an external device according to an embodiment of the present invention.

FIG. 5 is a simplified exemplary diagram illustrating communications between an implantable medical device and an external device according to another embodiment of the present invention.

### DETAILED DESCRIPTION OF THE INVENTION

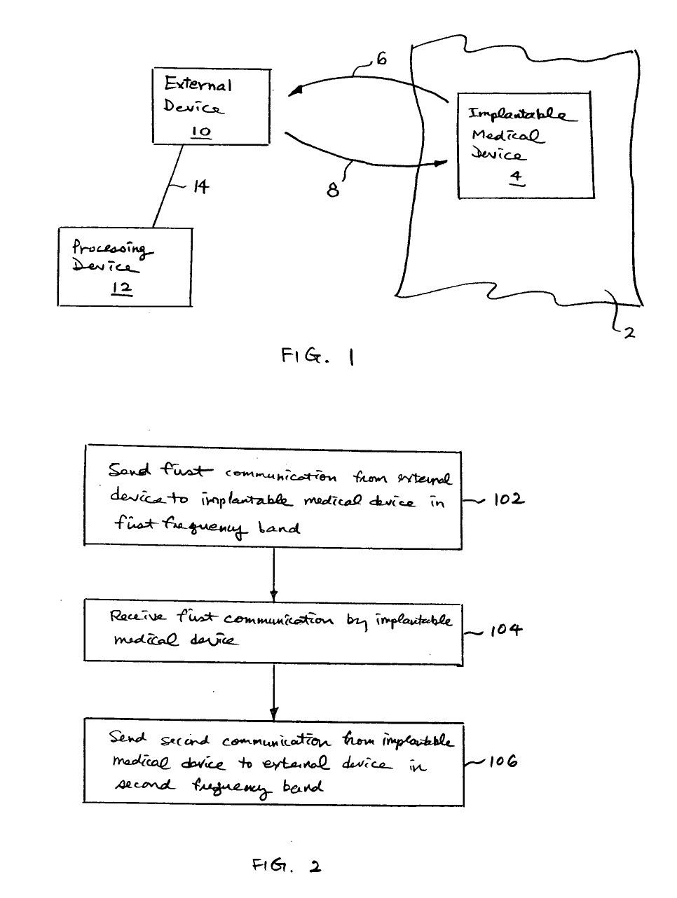

FIG. 1 is a simplified exemplary diagram showing wireless communications between an implantable medical device and an external device according to a specific embodiment of the invention. The body 2 of a patient has implanted therein an implantable medical device 4 to monitor physiological parameters and/or perform other functions within the body 2. The implantable medical device may be an electrocardiogram (ECG) device, a cardiac rhythm management device, a pacemaker, an endoscopic camera capsule, an implantable hearing device, or some other medical device that can be implanted within the patient's body 2. A surgical procedure may be used to insert the implantable medical device 4 within the patient's body 2.

An external device 10 is provided to interact with the implantable medical device 4. Wireless communication 8 can be transmitted in a first frequency band from the external device 10 to the implantable medical device 4. The first frequency band is the up link frequency band. The wireless communication 8 may be used to transmit programming information to reconfigure the implantable medical device 4, information modifying a therapy plan being performed by the implantable medical device 4, the selection of MICS band information for wireless communication 6 from the implantable medical device 4 to the external device 10, or the like.

FIG. 2 illustrates the communications between the implantable medical device 4 and the external device 10. In step 102, the external device 10 sends wireless communication 8 to the implantable medical device 4 via a transmitter or transceiver. In step 104, the implantable medical device 4 receives the wireless communication 8 from the external device 10 via a receiver or a transceiver. In step 106, the implantable medical device 4 transmits wireless communication 6, in a second frequency band different from the first frequency band, to the external device 10 via its transmitter or transceiver. The second frequency band is the down link frequency band. The wireless communication 6 may include, for example, physiological data monitored by the implantable medical device 4, status information about the implantable medical device 4, or an emergency medical event detected by the implantable medical device 4. In another example, the wireless communication 6 can be transmitted in the MICS band between 402-405 MHz. The external device 10 can further be coupled with a processing device 12 for further processing of the physiological data and/or other information received from the implantable medical device 4 or for printing or displaying of the received information. The external device 10 may be coupled to the processing device 12 through a wireless or physical link 14, such as a computer cable.

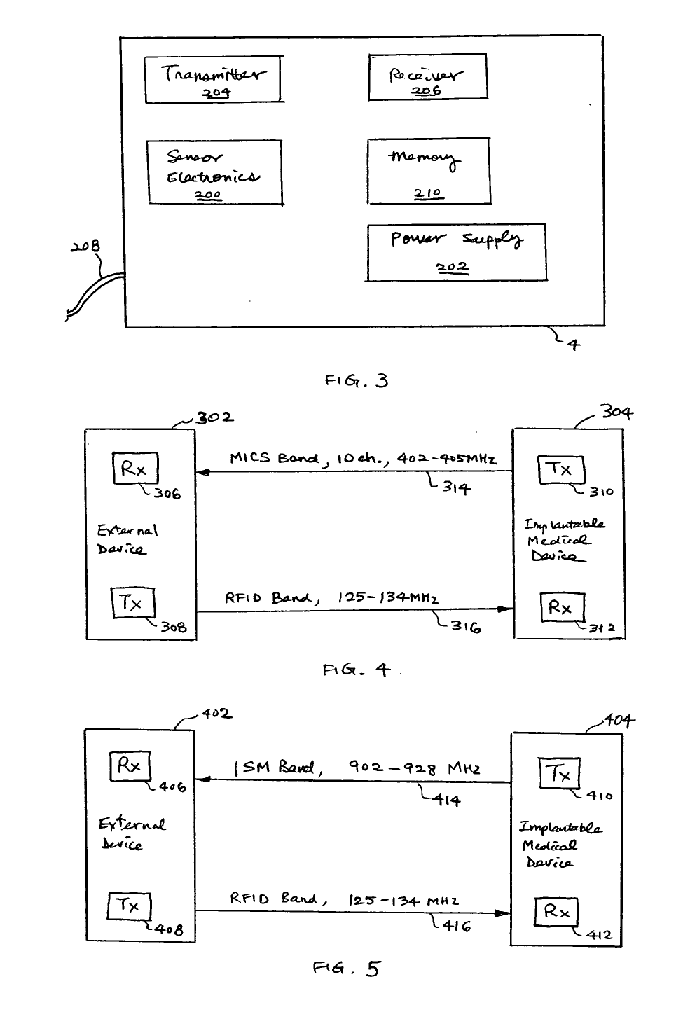

FIG. 3 is a simplified exemplary diagram of an implantable medical device according to a specific embodiment of the present invention. The implantable medical device 4 includes a number of different components contained within an external housing formed of a protective material designed to protect the components located within the external housing. For example, the protective material may be a lightweight plastic, titanium or epoxy material designed for implantation within the patient's body without any ill affects. A sensor 208 is provided for sensing any of a variety of the patient's physiological parameters, such as blood-oxygen saturation, pH, intracardiac temperature, and others. Of course, a plurality of sensors may be provided. The sensor 208 may be connected to the housing of the implantable medical device 4 or integrated directly within the housing of the implantable medical device 4. Sensor electronics 200 receive the informed sensed by the sensor 108, and further process and convert the signal into a usable form that can be easily stored or further transmitted to an external device. Examples of signal processing provided by the sensor electronics 200 include digitizing the received parameters, providing time contracted readback from one or multiple sensor signals, “chopping” multiple streams from the sensor together to form one output signal, and the like. A memory 210 may be provided for storage of the processed signals within the implantable medical device 4. The memory 210 may include a flash memory device or other solid-state memory storage device with a reduced form factor. The implantable medical device 4 further includes a power supply 202, a transmitter 204, and a receiver 206. The power supply 202 is typically a battery, but may be some other type of power supply. In some cases, the transmitter and the receiver may be formed as a transceiver.

The receiving and sending of wireless communications to and from the wireless device represent a significant portion of the power consumption of the implantable medical device 4. For this reason, the receiver 206 contained within the implantable medical device 4 is desirably an ultra-low power receiver that receives transmissions from the external device 10 at a lower frequency. The low frequency is lower than about 1 MHz. For example, the frequency used may be an RFID frequency between about (125-134) KHz. By using a lower frequency, the amount of current used in the implantable medical device 4 can be reduced and a reduced amount of power is consumed during operation. For example, the amount of current being consumed by the ultra-low power receiver 206 is about 2-3 μA. Conventional implantable devices utilize a current of 3-4 mA, nearly 3 orders of magnitude greater than that consumed within an exemplary embodiment of the invention. In one example, receiver 206 may receive transmissions as low as about 20-30 kHz. Due to the reduced amount of current being consumed, power management schemes such as sleep states or duty cycling do not need to be implemented for proper functioning of implantable medical device 4. Instead, implantable medical device 4 can be continuously left in an “ON” state during operation with a battery life equal or surpassing that of conventional implementations. This additionally improves the latency or response time of the implantable medical device 4 in that the device does not need to be powered on in response to a wake-up signal or duty cycled between on/off states. By removing the need for duty cycling or a sleep state, the circuitry of the implantable medical device can be simplified and reduced in size.

One additional reason that a low frequency is advantageous for transmission to the implantable medical device 4 is that it minimizes reduction of the signal due to body attenuation. The signal propagation characteristics of the patient's body tend to reduce the signal strength as the wireless communication from the external device 10 must pass through the patient's body before being received by implantable medical device 4. Lower frequency transmissions tend to undergo a smaller loss in signal strength due to body attenuation than higher frequency transmissions.

The transmitter 204 is used by the implantable medical device 4 to transmit wireless communications to the external device 10. For example, the transmitter 204 can be used to send wireless communications 6 in an asymmetrical pattern with a second frequency band different from the first frequency band of the wireless communication 8 received by the low-power receiver 206. A higher frequency band can be used to send the transmission from the transmitter 204 as the power consumption requirements for the receiver located in the external device 10 are not as stringent as those of the implantable medical device 4. The external device 10 may be of a larger size than the implantable medical device 4, thus allowing for larger and more powerful batteries to be used. Alternatively, an external power supply can be used to power the external device 10. The frequency band is typically higher than about 200 MHz. In one example, the frequency band being used by the transmitter 204 is the MICS band between 402 and 405 MHz. In another example, the frequency band being used by the transmitter 204 is between about 902 and 928 MHz.

FIG. 4 is a simplified exemplary diagram illustrating communications between an implantable medical device 304 and an external device 302 according to a specific embodiment of the present invention. The external device 302 includes a receiver 306 and a transmitter 308. The implantable medical device 304 includes a transmitter 310 and a low-power receiver 312. A low frequency transmission 316 is sent from the transmitter 308 to the low-power receiver 312. The frequency band used for the transmission 316 is below about 1 MHz, and may be in the RFID frequency band between about 125 and about 134 kHz In a specific embodiment, the information sent from the transmitter 308 may include a selection of a frequency in the MICS band for the transmitter 310 to use in a transmission 314 from the transmitter 310 to the receiver 306. The transmission 314 may be a transmission in the MICS band within one of ten channels between 402 and 405 MHz.

FIG. 5 is a simplified exemplary diagram illustrating communications between an implantable medical device 404 and an external device 402 according to another embodiment of the present invention. The external device 402 includes a receiver 406 and a transmitter 408. The implantable medical device 404 in this example is an ECG device that includes a transmitter 410 and a low-power receiver 412. A low frequency transmission 416 is sent from the transmitter 408 to the low-power receiver 412. The frequency band used for the transmission 316 is below about 1 MHz, and may be in the RFID frequency band between about 125 and about 134 kHz. A transmission 414 from the transmitter 410 to the receiver 406 may be a transmission in the Industrial, Scientific and Medical (ISM) band between about 902 and about 928 MHz commonly used for ECG devices. In another example other frequency range may be utilized instead.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

What is claimed is:

1. An implantable medical device to be implanted into a body of a patient, the implantable medical device comprising:
one or more sensors configured to monitor physiological condition of the body of the patient;
sensor electronics configured to process a signal received from the one or more sensors;
a receiver configured to receive information from the external device in a first frequency band;
a transmitter configured to sending information to an external device using a second frequency band which is different from the first frequency band; and
a power supply;
wherein the receiver is powered on continuously when the implantable medical device is implanted into the body of the patient.

2. The device of claim 1 wherein the receiver is an ultra-low power receiver.

3. The device of claim 2 wherein the ultra-low power receiver uses a current of about 3 microamps or less.

4. The device of claim 2 wherein a receiving range of the receiver is at least about 2 meters.

5. The device of claim 1 wherein the one or more sensors comprise ECG sensors.

6. The device of claim 1 wherein the second frequency band is higher than the first frequency band.

7. The device of claim 1 wherein the first frequency band is an RFID frequency in the range of about 125 to about 134 kHz.

8. The device of claim 7 wherein the second frequency band is a MICS band in the range of 402-405 MHz.

9. The device of claim 7 wherein the second frequency band is in the range of about 902 to about 928 MHz.

10. The device of claim 1 wherein the information received by the receiver from the external device comprises a selection of a frequency in a MICS band for the transmitter to send information to the external device.

11. The device of claim 1 wherein the receiver and transmitter are integrated as a transceiver.

12. A method for communicating with an implantable medical device to be implanted into a body of a patient, which includes one or more sensors configured to monitor physiological condition of the body of the patient and sensor electronics configured to process a signal received from the one or more sensors, the method comprising:
receiving a first communication from an external device by a receiver in the implantable medical device within a first frequency band;
sending a second communication from the implantable medical device to the external device within a second frequency band which is different from the first frequency band; and
providing a power supply in the implantable medical device,
wherein the receiver is powered on continuously when the implantable medical device is implanted into the body of the patient.

13. The method of claim 12 wherein the first communication is received by a receiver in the implantable medical device, the receiving being an ultra-low power receiver.

14. The method of claim 13 wherein the ultra-low power receiver uses a current of about 3 microamps or less.

15. The method of claim 13 wherein a receiving range of the receiver is at least about 2 meters.

16. The method of claim 12 further comprising sensing ECG signals in the body of the patient.

17. The method of claim 12 wherein the second frequency band is higher than the first frequency band.

18. The method of claim 12 wherein the first frequency band is an RFID frequency in the range of about 125 to about 134 kHz.

19. The method of claim 18 wherein the second frequency band is a MICS band in the range of 402-405 MHz.

20. The method of claim 18 wherein the second frequency band is in the range of about 902 to about 928 MHz.

21. The method of claim 12 wherein the information received by the receiver from the external device comprises a selection of a frequency in a MICS band for the transmitter to send information to the external device.

22. The method of claim 12 wherein the implantable medical device includes a transceiver to receive the first communication and send the second communication.

23. An implantable medical device to be implanted into a body of a patient, the implantable medical device comprising:
one or more sensors configured to monitor physiological condition of the body of the patient;
sensor electronics configured to process a signal received from the one or more sensors;
a receiver configured to receive information from the external device in a first frequency band;
a transmitter configured to sending information to an external device using a second frequency band; and
a power supply;
wherein the receiver is powered on continuously when the implantable medical device is implanted into the body of the patient; and
wherein the first frequency band is lower than about 1 MHz.

24. The device of claim 23 wherein the first frequency band is an RFID frequency in the range of about 125 and about 134 kHz.

25. The device of claim 23 wherein the second frequency band is higher than the first frequency band.

26. The device of claim 25 wherein the second frequency band is higher than about 200 MHz.
